(19) United States
(12) Patent Application Publication
(10) Pub. No.: US 2019/0105385 A1
(43) Pub. Date: Apr. 11, 2019

(54) A COMBINATION VACCINE AGAINST PCV2 VIRUS AND MYCOPLASMA HYOPNEUMONIAE INFECTION

(71) Applicant: Intervet Inc., Madison, NJ (US)

(72) Inventors: Melanie Sno, Venlo (NL); Maarten Hendrik Witvliet, Oostrum (NL); Vicky Fachinger, Bad Soden (DE)

(73) Assignee: Intervet Inc., Madison, NJ (US)

(21) Appl. No.: 16/086,690

(22) PCT Filed: Mar. 22, 2017
(86) PCT No.: PCT/EP2017/056831

§ 371 Date: Sep. 20, 2018

(30) Foreign Application Priority Data
Mar. 23, 2016 (EP) 16161997.8

(51) Int. Cl. A61K 39/12; A61K 39/02; A61P 31/12; A61P 31/04

(52) U.S. Cl. CPC A61K 39/12; A61K 39/0241; A61K 2039/521; A61P 31/12; A61P 31/04; A61K 39/105

(57) ABSTRACT

The present invention pertains to a vaccine comprising in combination non-replicating immunogen of porcine circo virus type 2 and non-replicating immunogen of Mycoplasma hyopneumoniae and an adjuvant containing a nano-emulsion of mineral oil in water, for use in prophylactically treating an animal against an infection with porcine circovirus type 2 (PCV2) and an infection with Mycoplasma hyopneumoniae by administration of the vaccine into the dermis of the animal.

### GENERAL FIELD OF THE INVENTION

The invention in general pertains to the field of swine health. Swine are prone to many pathogenic micro-organisms. Control of infection is commonly done by stable and feed management, treatment with pharmaceuticals such as anti-viral drugs and antibiotics, or prophylactic treatment using vaccines. In particular, the invention pertains a vaccine against porcine circo virus type 2 (PCV-2) and Mycoplasma hyopneumoniae infection, and to a method of protecting an animal against such infections using the vaccine.

### BACKGROUND ART

The most important pathogens that give rise to significant economic losses in the swine industry are PCV2 (porcine circovirus type 2), Mycoplasma hyopneumoniae, PRRS (porcine reproductive and respiratory syndrome) virus and Lawsonia intracellularis.

PCV-2 is linked to the post-weaning multisystemic wasting syndrome (PMWS) observed in young pigs. This disease was encountered for the first time in Canada in 1991. The clinical signs and pathology were published in 1996, and include progressive wasting, dyspnea, tachypnea, and occasionally icterus and jaundice.

Nayar et al., Can. Vet. J. Volume 38, June 1997 detected porcine circo virus in pigs with clinical symptoms of PMWS and concluded that a PCV, other than the known PCV recognized as a natural inhabitant of PK-15 cells, could be linked to PMWS. Later publications (Hamel et al., J. Virol., 72(6), 5262-5267, 1998; Meehan et al., J. gen. Virol., 79, 2171-2179, 1998) confirmed these findings, and it was proposed (Meehan et al., supra) to refer to the new pathogenic PCV as PCV-2, whereas the original PK-15 cell culture isolate (Tischer et al., Nature 295, 64-66, 1982), should be referred to as PCV-1. PCV-2 is a small (17-22 nm) icosahedral non-enveloped virus containing a circular [symbol] 2 isolates originating from different regions in the world seem to be closely related to each other and display 95 to 99% nucleotide sequence identities (Fenaux et al., J. Clin. Micorbiol., 38(7), 2494-2503, 2000). ORF-2 of PCV encodes the capsid protein of the virus. The ORF 2 of PCV 2 encodes a protein of about 233 amino acids. The ORF 2 of all PCV-2 isolates share 91-100% nucleotide sequence identity and 90-100% deduced amino acid sequence identity.

Mycoplasma hyopneumoniae (Mhyo) is a species of bacteria known to cause the disease Porcine Enzootic Pneumonia, a highly contagious and chronic disease affecting pigs. Mhyo is small in size (400-1200 nm), has a small genome (893-920 kilo-base pairs (kb)) and lacks a cell wall. Mhyo attaches to the cilia of epithelial cells in the lungs of swine. They cause cilia to stop beating, clumping and loss of cilia, eventually leading to epithelial cell death. This is the source of the lesions found in the lungs of pigs with porcine enzootic pneumonia. This damage impedes normal ciliary clearance and often secondary infections develop. This causes a significant reduction in the growing weight of the animals. Losses in the U.S.A. have been previously estimated to be up to 1 billion dollars per annum. Porcine enzootic pneumonia is endemic worldwide and Mhyo is present in almost every pig herd. The immune response caused by the presence of Mhyo in pigs is slow and ineffective. Treatment of this disease is therefore of the utmost importance but is limited to antibiotics, which are currently only partly effective as they do not completely remove the infection. Vaccines have been found to reduce the severity of the disease but do not completely prevent the disease from occurring in infected pigs.

PRRS virus first reported in 1987 in North America and Central Europe. PRRS virus is a small, enveloped RNA virus. It contains a single-stranded, positive-sense, RNA genome with a size of approximately 15 kilobases. The genome contains nine open reading frames. The virus is a member of the genus Arterivirus, family Arteriviridae, order Nidovirales. The two prototype strains of PRRSV are the North American strain, VR-2332, and the European strain, the Lelystad virus (LV). The European and North American PRRSV strains cause similar clinical symptoms. In the early 2000s a highly pathogenic strain of the North American genotype emerged in China. This strain, HP-PRRSV, is more virulent than all other strains, and causes great losses in Asian countries worldwide. For any PRRS virus, subclinical infections are common, with clinical signs occurring only sporadically in a herd. Clinical signs include reproductive failure in sows such as abortions and giving birth to stillborn or mummified fetuses, and cyanosis of the ear and vulva. In neonatal pigs, the disease causes respiratory distress, with increased susceptibility to respiratory infections such as Glasser's disease.

Lawsonia intracellularis causes proliferative enteropathy, also known as ileitis, which is a common enteric disease of post-weaned pigs worldwide. The characteristic lesion is a proliferation of immature enterocytes in the ileal intestinal crypts; these cells usually contain the causative bacteria within their apical cytoplasm. At autopsy, histologic lesions can be confirmed as Lawsonia-positive by visualization of 1.5-2.5 μm long, vibrioid shaped bacteria especially in enterocytes, but also often within macrophages located in the lamina propria between crypts, and in mesenteric lymph nodes. Clearance of the bacteria from the enterocytes leads to resolution of the associated proliferative lesions, indicating a direct local effect of the bacteria on the crypts. The presence of Lawsonia intracellularis in these lesions has been demonstrated using PCR, both in animals manifesting disease as in animals manifesting only subclinical infection. Clinical cases are usually present in the grower-finisher period; in some older finisher pigs an acute hemorrhagic form has been recorded.

Vaccines against the above identified pathogens are commonly known. A conventional vaccine to prophylactically treat animals, in particular pigs, against an infection with PCV 2, may be based on whole inactivated PCV-2 virus as (non-replicating) immunogen. Also, in the art it has been shown that the ORF2 encoded capsid protein (e.g. when recombinantly expressed) is suitable as a subunit immunogen of porcine circo virus type 2 for use in an adequate vaccine. This can be understood since this subunit, in a circulatory system, shows up the same way as the virus itself, essentially differing in the fact that the DNA and non-structural proteins are not present inside the capsid. In the art several vaccines against PCV2 are commercially available. Porcilis® PCV (available from MSD Animal Health, Boxmeer, The Netherlands) is a vaccine for protection of pigs against porcine circo virus type 2, for use in pigs from three weeks and older. When given as a two-shot (two dose) vaccine, the duration of immunity (DOI) is 22 weeks, almost completely covering the fattening period of pigs. Ingelvac CicroFlex® (available from Boehringer Ingelheim, Ingelheim) is a vaccine for protection of pigs against porcine circo virus type 2, for use in pigs from two weeks and older. It is registered as a one-shot (one dose) vaccine only. Circovac® (available from Merial, Lyon, France) is a vaccine for protection of pigs against porcine circo virus type 2, for use in pigs three weeks and older. Suvaxyn® PCV (available from Zoeitis, Capelle a/d Ussel, The Netherlands) is a vaccine for protection of pigs against porcine circo virus type 2, for use in pigs from three weeks and older. Other PCV2 vaccines are described for example in WO2007/028823, WO 2007/094893 and WO2008/076915.

Regarding Mycoplasma hyopneumoniae many commercial vaccines exist and these are routinely used in the majority of commercial swine farming operations. Generally these vaccines comprise non-replicating immunogens such as subunit proteins and/or bacterins (i.e. a composition comprising killed bacteria, either as whole cells, (partly) lysed, homogenised, French pressed, a combination of this or comprising the killed bacteria in another form as long as the composition is derived from a killed bacterial culture) which are typically administered by parenteral injection. Some examples are: RespiSure® (Zoetis), Ingelvac® M. hyo, and MycoFLEX® (Boehringer Ingelheim), Hyoresp® (Merial), Stellamune® Mycoplasma (Elanco Animal Health), Fostera® PCV MH (Zoetis) and M+Pac® (MSD Animal Health).

Regarding PRRS virus, although inactivated virus vaccines have been described and are commercially available, modified Live Vaccines (MLV) vaccines comprising either the European type (type I) or the North American type (type II) in live attenuated form, are the primary immunological tool for its control. Several vaccines are commercially available in the art. Porcilis® PRRS (available from MSD Animal Health, Boxmeer, The Netherlands) is a vaccine comprising live attenuated PRRS virus type I and is registered to reduce infection (viraemia) caused by infection with PRRS virus. Ingelvac PRRS® MLV (available from Boehringer Ingelheim, Ingelheim) is a vaccine that aids in the reduction of disease caused by PRRS virus and which vaccine provides cross protection against strains of different types. Fostera® PRRS (available from Zoeitis, Florham Park, N.J., USA) is also a MLV vaccine and is registered for protection against both the respiratory and reproductive forms of disease caused by PRRS virus. Other PRRS vaccines are described for example in WO2006/074986, U.S. Pat. No. 8,728,487 and WO2014/048955.

Vaccines to combat Lawsonia intracellularis by inducing active protection are commercially available and described in the art. These vaccines are available under the tradenames Enterisol® Ileitis (Boehringer Ingelheim Vetmedica, USA) which is a live attenuated vaccine, and Porcilis® Ileitis (Merck Animal Health, USA) which is a vaccine comprising non-replicating immunogen of Lawsonia intracellularis in the form of a bacterin.

### OBJECT OF THE INVENTION

There is a continuous need for convenient, safe and efficacious means for the management of swine health. The object of the invention is to provide a vaccine that meets this need, in particular the need for a novel PCV2/Mhyo combination vaccine.

### SUMMARY OF THE INVENTION

In order to meet the object of the invention, a new vaccine has been devised, the vaccine comprising in combination non-replicating immunogen of porcine circo virus type 2 and non-replicating immunogen of Mycoplasma hyopneumoniae and an adjuvant containing a nano-emulsion of mineral oil in water, for use in prophylactically treating an animal against an infection with porcine circovirus type 2 (PCV2) and an infection with Mycoplasma hyopneumoniae by administration of the vaccine into the dermis of the animal.

Although for both pathogens vaccines are known and commercially available, there is no combination vaccine available for intradermal administration, which vaccine is efficacious and at the same time safe for use in young animals. As is commonly known, not all combinations of antigens contemplated or suggested may lead to a safe and effective combination vaccine. In fact, there is a high level of uncertainty with regard to the stability, safety and efficacy of the combination vaccine for use at a particular administration site, even when the single (monovalent) vaccines are safe and efficacious or a combination vaccine is known for use at an alternative administration site. It was surprisingly found that to arrive at a safe and effective combination vaccine for intradermal administration, use could be made of an adjuvant composition that contains a nano-emulsion of mineral oil in water. The reason for the surprise was that this type of emulsion is typically regarded as unsafe when used for intramuscular administration. This is known i.a. from the publication by scientists of the adjuvant developing and producing firm SEPPIC (Deville et al in Revue Med. Vet, 2009, 160, 11, 514-519) which shows in table II that with the use of the nano-emulsion Montanide® IMS 251, there are adverse pyrogenic effects leading to an average temperature increase of the pigs of 2° C., 4 hours after vaccination (compared to −0.8 to 1.5° C. for any of the other 5 formulations tested). An increase of 2° C. is well above the 1.5° C. that is allowed according to European Pharmacopoeia monograph 2448 for an Mhyo vaccine, which might explain why a nano-emulsion of mineral oil is not commercially used for any swine vaccine.

Regarding any combination vaccine in general, the committee for veterinary medicinal products of the European Agency for the Evaluation of Medicinal Products (EMEA) in its publication “Note for guidance: requirements for combined veterinary products” (EMEA, 2000, CVMP/IWP/52/97-FINAL), stated (page 2/6) that the “development of combined vaccines is not straightforward. Each combination should be developed and studied individually in terms of quality, safety and efficacy”. The committee further indicates that the search for a good combination vaccine typically includes the compatibility between the individual components in the combined vaccine, including for example preservatives, excipients and stabilisers, inactivating agents and adjuvants. On page 3, top paragraph, it is stated that “In combined vaccines, the presence of more than one component can often cause an interaction, leading to either a diminished or an increased response to individual components, compared to when the specific component(s) is administered alone . . . . Such interactions are often immunological in nature, but may also be caused by other factors with less direct effects on the immune system”, and also “When an adjuvant is used to augment the immune response to a combined vaccine, special problems may appear.”

The U.S. Department of Health and Human Services, Food and Drug Administration, Center for Biologics Evaluation and Research, published in April 1997 a “Guidance for Industry, for the evaluation of combination vaccines for preventable diseases: Production, Testing and Clinical Studies”, in which guidance it is stated (page 3, under “Compatibility of Components”) that “Experience has shown that combining monovalent vaccines may result in a new combination which is less safe or effective than desirable. Sometimes the components of inactivated vaccines may act adversely on one or more of the active components”, indicating that especially an inactivated vaccine may negatively influence the efficacy of a live vaccine, such as for example occurred when combining a live pertussis vaccine and an inactivated poliovirus vaccine that resulted in a vaccine with decreased pertussis potency. It is indicated that any additional components in the vaccine might complicate the safety and potency of the final product when compared to the individual vaccines.

The World Health Organization (WHO) has published an e-learning course called “Vaccine Safety Basics”, which in the MODULE 2 contemplates combination vaccines.

This module starts with “Licensed combination vaccines undergo extensive testing before approval by national authorities to assure that the products are safe, effective, and of acceptable quality.” It is also stated that “With all combinations, manufacturers must therefore evaluate the potency of each antigenic component, the effectiveness of the vaccine components when combined to induce immunity, risk of possible reversion to toxicity, and reaction with other vaccine components.”

It is thus not straightforward to devise a new combination vaccine, let alone a new vaccine for a particular site of administration. The World Health Organization (WHO) for example has published an e-learning course called “Vaccine Safety Basics”, in which course on page 53 it is reported that “The route of administration is the path by which a vaccine (or drug) is brought into contact with the body. This is a critical factor for success of the immunization. A substance must be transported from the site of entry to the part of the body where its action is desired to take place. Using the body's transport mechanisms for this purpose, however, is not trivial.”

In this respect the California Department of Health Services' Immunization Branch has published guidelines for correct immunization (http://www.cdc.gov/vaccines/pubs/pinkbook/downloads/appendices/d/vacc_admin.pdf). With regard to the administration site it is stated on page 7, first full paragraph that “The recommended route and site for each vaccine are based on clinical trials, practical experience and theoretical considerations. This information is included in the manufacturer's product information for each vaccine. There are five routes used in the administration of vaccines. Deviation from the recommended route may reduce vaccine efficacy or increase local adverse reactions.” On page 14 the only US-licensed intradermal vaccine is addressed: “Fluzone Intradermal is the only U.S.-licensed vaccine that is administered by the intradermal route. It is approved only for use in persons 18 through 64 years of age. This Fluzone formulation is not the same as intramuscular formulations of inactivated influenza vaccine (TIV). Other TIV formulations should NOT be administered by the intradermal route.”

All in all, it is commonly known that vaccination at a particular site, let alone vaccination of a combination vaccine at a particular site, is not straightforward and requires experimentation to determine safety and efficacy.

Regarding intradermal administration, although intradermal administration can be carried out using a needle-less vaccination device such as the IDAL® vaccinator (available from MSD Animal Health, Boxmeer, The Netherlands), “intradermal” administration per se should not be equated with “needle-less” administration. The World health Organization in its Aug. 27, 2009 paper titled “Intradermal Delivery of Vaccines; A review of the literature and the potential for development for use in low- and middle-income countries” indeed clearly indicates that “needle-less” vaccination does not necessarily mean “intradermal” vaccination (see Table 1, Page 3 of the review). Only when a needle-less device is “configured for intradermal vaccination”, then a vaccine may indeed be delivered (at least partly) into the dermis. Otherwise the vaccine may be delivered subcutaneous or intramuscularly in its entirety.

The present invention also pertains to a method for prophylactically treating an animal against an infection with porcine circovirus type 2 (PCV2) and an infection with Mycoplasma hyopneumoniae by intradermally administrating to the animal a vaccine comprising in combination non-replicating immunogen of PCV2, non-replicating immunogen of Mycoplasma hyopneumoniae, and an adjuvant containing a nano-emulsion of mineral oil in water, and to the use of non-replicating immunogen of porcine circo virus type 2 (PCV2) and non-replicating immunogen of Mycoplasma hyopneumoniae to manufacture a vaccine comprising in combination the immunogen of PCV2, non-replicating immunogen of Mycoplasma hyopneumoniae, and an adjuvant containing a nano-emulsion of mineral oil in water, for intradermal administration to an animal to prophylactically treat the animal against an infection with PCV2 and an infection with Mycoplasma hyopneumoniae.

It is noted that in a vaccine the immunogen (also called antigen) is typically combined with a pharmaceutically acceptable carrier, i.e. a biocompatible medium, viz. a medium that after administration does not induce significant adverse reactions in the subject animal, capable of presenting the immunogen to the immune system of the host animal after administration of the vaccine, such as a liquid containing water and/or any other biocompatible solvent or a solid carrier such as commonly used to obtain freeze-dried vaccines (based on sugars and/or proteins), optionally comprising immunostimulating agents (adjuvants), which upon administration to the animal induces an immune response for treating an animal against an infection with a wild-type micro-organism, i.e. for aiding in preventing, ameliorating or curing such infection or a disorder arising therefrom. Optionally other substances such as stabilisers, viscosity modifiers or other components are added depending on the intended use or required properties of the vaccine.

### Definitions

A vaccine is a pharmaceutical composition that is safe to administer to a subject animal, and is able to induce protective immunity in that animal against a pathogenic micro-organism, i.e. to induce a successful prophylactic treatment as defined here below.

Non-replicating immunogen of a pathogen is any substance or compound corresponding to the pathogen, other than the live replicating pathogen as a whole (either in wild type of attenuated form), against which pathogen an immunological response is to be elicited, such that the corresponding virulent pathogen or one or more of its virulence factors will be recognized by the host's immune system as a result of this immune response and are ultimately at least partly neutralized. Typical examples of non-replicating immunogens are killed whole pathogens and subunits of these pathogens such as capsid proteins and surface expressed proteins, for example recombinantly expressed proteins.

A live attenuated pathogen is a viable, replication competent (viable) form of the pathogen having reduced virulence. The process of attenuation takes an infectious pathogen and alters it so that it becomes harmless or less virulent, typically by either multiple passages of the pathogen through cell systems or by genetically modifying the pathogen.

Prophylactic treatment against an infection with a pathogen is aiding in preventing or ameliorating an infection with that pathogen or a disorder arising from that infection, resulting from a post treatment challenge with a pathogenic pathogen, in particular to reduce its load in the host after such challenge and optionally to aid in preventing or ameliorating one or more clinical manifestations resulting from the post treatment infection with the pathogen.

A nano-emulsion is an emulsion of particles having a volume mean average particle size below 300 nm (the wave-length of visible light) such that the emulsion, at a concentration of 5 vol. % dispersed oil, is translucent to opalescent, as opposed to a micro-emulsion which is white (like milk) at 5 vol. % dispersed oil. In a nano-emulsion, typically more than 90% of the particles have a particle size below 300 nm, with the peak value for the (volume) mean particle size typically being between 20 and 200 nm.

Single dose administration of a vaccine for use in prophylactically treatment means that in order to arrive at protective immunity, the vaccination does not need to be boosted with a second administration of the vaccine. In a two-shot regime, the first (prime) vaccination is typically boosted within 6 weeks from the first administration, commonly within 3 or even 2 weeks from the first administration, and only after the second (boost) administration protective immunity, i.e. a successful prophylactic treatment as defined here above, may be obtained.

### EMBODIMENTS OF THE INVENTION

In a first embodiment the vaccine is administered by a single dose. It was found that a single dose administration led to an effective vaccine. This provides for a very convenient and economical way to protect animals against both pathogens.

In a next embodiment the vaccine is administered with a needle-less vaccination device, using a jet of the vaccine to reach the dermis through the skin of the animal. Vaccination into the dermis is in this embodiment provided by a needle-less vaccination device using a liquid jet of the vaccine (a high pressurized fluid stream), typically using a very low volume of vaccine in the range of 0.05 to 0.2 ml. This further increases the safety of the vaccine and method of administration.

In another embodiment the non-replicating immunogen is recombinantly expressed ORF2 protein of porcine circo virus type 2, for example expressed by baculo virus as known in the art. This recombinant protein has proven to be suitable for application in the present invention. In particular, the ORF2 protein can be expressed in a baculo virus expression system such as described in WO2007/028823, WO 2007/094893 or WO2008/076915.

In yet another embodiment the non-replicating immunogen of Mycoplasma hyopneumoniae is a bacterin. Such Mhyo antigen is relatively easy to produce and has a good track record of efficacy in the everyday swine industry practice.

In still another embodiment the vaccine in addition comprises live attenuated PRRS virus. In this embodiment the vaccine is capable of providing protection against three major swine pathogens by using just one vaccine.

In again another embodiment the live attenuated PRRS virus is combined in the vaccine within 24 hours, preferably within 6 hours before administration. Combining the antigens right before administration provides more freedom to choose the excipients since long-term stability, although known for many pharmaceutical compositions, even for combination vaccines including PCV2 ORF2 antigen (for example Porcilis® PCV M Hyo, available from MSD Animal Health), as such is known, might still not be straightforward to achieve, at least not for any and all pharmaceutically acceptable carrier compositions.

In another embodiment the vaccine in addition comprises non-replicating immunogen of Lawsonia intracellularis. In this embodiment the vaccine is capable of providing protection against three major swine pathogens by using just one vaccine. Corresponding to what was said herein before regarding the addition of PRRS immunogen to the vaccine, the immunogen of Lawsonia intracellularis in an embodiment is combined with the immunogen of PCV2 and Mycoplasma hyopneumoniae within 24 hours before administration, preferably within 6 hours before administration. In a further embodiment the immunogen of Lawsonia intracellularis is added to the vaccine in the form of a freeze-dried composition of Lawsonia intracellularis bacterin.

The invention will now be explained further using the following examples.

### EXAMPLES

Study 1

Objective

The objective of this study was to assess the safety and efficacy of different experimental PCV2/Mhyo combination vaccines for intradermal application in piglets.

Experimental Design

The progeny of approximately 10 sows were available for this study. The animals had maternally derived antibodies (MDA) against PCV2. A total of 50 piglets were allotted to 4 treatment groups of 10 piglets each and 2 control groups of 5 piglets each. PCV2 antigen was produced by two different concentration processes, a first one using ultrafiltration (UF) and a second one using gravitational forces (G). Vaccines were formulated by the addition of two different adjuvants using the commercially available micro-emulsion adjuvant XSolve (average particle size slightly below 0.5 μm; obtainable from MSD Animal Health, Boxmeer, The Netherlands), differing in final concentration of the oil. XSolve 50 contained 20.8% mineral oil and X-solve 30 contained 12.5% mineral oil.

Piglets from groups 1 to 4 were intradermally vaccinated with a single dose of 0.2 ml vaccine, applied with the IDAL® vaccinator (MSD Animal Health) when they were approximately 3 weeks old. Piglets from groups 5 and 6 were injected with a placebo in the same manner. The combination vaccine contained PCV2 ORF2 protein at a final concentration of 9 μg per dose and Mhyo bacterin at 1 PCVU/dose (the same as in the commercially available vaccine Porcilis® M Hyo ID ONCE of MSD Animal Health). Here below the different groups are indicated:

Group 1: PCV/Mhyo, XSolve 30, UF

Group 2: PCV/Mhyo, XSolve 50, UF

Group 3: PCV/Mhyo, XSolve 30, G

Group 4: PCV/Mhyo, XSolve 50, G

Group 5: Placebo, XSolve 30

Group 6: Placebo, XSolve 50

All piglets were observed daily for clinical signs. On the day of vaccination clinical signs were monitored in all animals one and four hours post vaccination. The body temperature was taken from all animals, daily from day −1 until day +4, and at 4 hours post vaccination. Local reactions were monitored by palpation starting on the day of vaccination until 4 weeks after vaccination or until regression of the reaction. Blood samples were collected from all animals the day before vaccination, and 2, 3, 5 and 10 weeks after vaccination. Serum samples from each animal taken before vaccination and 2, 3, 5 and 10 weeks thereafter were tested for antibodies against Porcine Circovirus Type 2 (PCV2).

Results

At the start of the experiment, all animals were found to be healthy.

At the time of vaccination and onwards from one day after vaccination all groups had comparable temperatures. At four hours after vaccination all groups vaccinated with a combination vaccine showed a considerable increase in body temperature when compared to the control groups. No significant differences were seen between the UF and G groups. The mean body temperature increase in the vaccinated groups was up to 1.0° C. higher than the mean body temperature in the control animals. Peak values were well above 41° C. (up to 41.6° C.) whereas values in the control groups were in the 39-40° C. range, which peak values of 40.2° C.

Regarding local reactions as assessed by palpation, in the control groups, no animals in the XSolve 30 group had any local reactions. In the XSolve 50 group, 60% of the animals had local reactions but the maximum size stayed below 1.5 cm. For the vaccinated groups the situation was completely different. Of the animals receiving the XSolve 30 vaccine, 60-90% showed local reactions, with maximum sizes around 5 cm. In the XSolve 50 groups 70-80% of the animals showed local reactions, with maximum sizes around 6 cm, the local reactions being blue and/or painful to the animal.

Although serological responses to the vaccines were good (results not shown), the vaccines were generally regarded as not being safe in particular due to severe local reactions.

Study 2

Objective

With respect to Study 1, the objective of this second study was to assess whether or not the combination vaccines could be made safer when lowering the amount of the micro-emulsion, while retaining efficacy, and also to test other potential adjuvants with the aim at arriving at a safe and efficacious combination vaccine.

Experimental Design

A total of 60 piglets was allotted to 4 treatment groups of 15 piglets each. The piglets were vaccinated when they were approximately three weeks old. Piglets from groups 1 to 3 were vaccinated with a single dose of 0.2 ml vaccine formulated with PCV2-Mhyo antigen as used in Study 1, using the XSolve adjuvant at a concentration of 5% for the mineral oil (XSolve 12), or in the alternative the same vaccine using in addition to the XSolve micro emulsion the commonly accepted alum adjuvant, or a vaccine using an adjuvant based on squalane (hydrogenated shark liver oil) adjuvant (0.2 ml). Piglets from group 4 were vaccinated with a single dose of Porcilis® Mhyo ID ONCE (0.2 ml). All piglets were vaccinated intradermally in the right side of the neck. Here below the different groups are indicated:

Group 1: PCV/Mhyo, XSolve 12

Group 2: PCV/Mhyo, XSolve 12 plus Alum (Al(OH)3

Group 3: PCV/Mhyo, Squalane

Group 4: Porcilis® M Hyo ID ONCE, positive control

On study day (SD) 29 all animals were challenge infected intratracheally on two consecutive days with 10 ml of a virulent Mhyo strain. On SD 52 or 53 all animals were necropsied.

All piglets were observed daily after vaccination for clinical signs. Local reactions were monitored by palpation every second day, starting on the day of vaccination until 29 days post vaccination, or until local reactions had resolved. Serum samples were collected from all animals on the day of vaccination as well as on SD 22, 29 and 52/53.

Samples were tested for antibodies against PCV2 and Mhyo and were compared with each other. At necropsy, the Mhyo specific lung lesions were scored.

Results

At the start of the experiment all animals were found to be healthy.

Following vaccination the severity of local reactions (average maximum size, number of animals with local reactions) was the lowest in the Group 1 (XSolve 12) and Group 3 (Squalane). In these groups the average maximum size of the local reactions was 1.1 and 0.9 cm respectively, while the percentage of animals with local reactions were 40 and 65%. In the other groups most animals had local reactions (90-100% of piglets) and the average maximum size was between 1.5 and 1.8 cm. All these figures are acceptable for vaccines authorised for use in practice.

Regarding PCV serology, at the time of vaccination (SDO) all animals were negative for PCV2 IgM antibodies. Following vaccination no clear difference in average PCV2 total Ig antibody response could be observed between the test groups 1, 2 and 3. Three weeks post vaccination the percentage of PCV2 IgM positive animals was 80-100% per test group and 0% for the controls. This shows that the combination vaccines were able to actively induce an anti-ORF2 antibody titer.

Regarding Mhyo, at the start of the experiment none of the piglets were positive for antibodies against Mhyo. Four weeks post vaccination (SD29) there was minimal response against Mhyo in the test groups 1 to 3. Only 1 animal in group 2 was positive. In the positive control group (Porcilis Mhyo ID Once) 10 animals (67%) of the piglets had a Mhyo antibody response. Following Mhyo challenge almost all animals had a positive Mhyo serological response. This indicated that the combination vaccines would not have an acceptable efficacy, which indication was confirmed by assessing the lung lesion scores which are summarised in Table 1.

TABLE 1
Median and mean percentage of Lung Lesions

| Group | Median | Mean |
|---|---|---|
| 1. PCV2/Mhyo Xsolve 12 | 2.20 | 4.69 |
| 2. PCV2/Mhyo Xsolve 12 + Al(OH)3 | 1.90 | 3.01 |
| 3. PCV2/Mhyo squalane | 3.99 | 5.15 |
| 4. Porcilis Mhyo ID Once (XSolve 50) | 0.90 | 1.97 |

In conclusion, by using a less concentrated micro emulsion of a mineral oil, optionally with the addition of an alum adjuvant, or by using squalane as an adjuvant, safe PCV2/Mhyo combination vaccines could be devised, but these vaccines were regarded as not sufficiently efficacious for combating an infection with Mycoplasma hyopneumoniae.

Study 3

Objective

The objective of the third study was to find an alternative adjuvant that could be used to make the PCV2/Mhyo combination vaccine safe for ID use, while at the same time retaining efficacy and optionally be suitable for admixing non-replicating immunogen of Lawsonia intracellularis.

Experimental Design

A total of 75 piglets was allotted to 5 treatment groups of 15 piglets each. The piglets were vaccinated when they were approximately three weeks old. Piglets from groups 1 to 3 were vaccinated with a single dose of 0.2 ml vaccine formulated with PCV2-Mhyo antigen as used in Study 1, using the nano-emulsion of mineral oil Montanide IMS251C (available from SEPPIC France) according to manufacturer's instructions. The vaccine for Group 1 contained a half dose of Mhyo antigen (0.5 PCVU/dose) as compared to the other groups. Before vaccination, freeze-dried Lawsonia bacterin (at a dose of approximately 10⁸ cells) was added to the vaccine for Group 3. Piglets from group 4 were vaccinated with a single dose of Porcilis® Mhyo ID Once (0.2 ml). All piglets were vaccinated intradermally in the right side of the neck. Piglets from group 5 were not vaccinated (negative control group). Here below the different groups are indicated:

Group 1: PCV/Mhyo, 0.5 PCVU Mhyo, Montanide

Group 2: PCV/Mhyo, 1 PCVU Mhyo, Montanide

Group 3: PCV/Mhyo, 1 PCVU Mhyo plus Lawsonia, Montanide

Group 4: Porcilis® M Hyo ID ONCE, positive control

Group 5: No vaccination, negative control

On study day (SD) 35 all animals were challenge infected intratracheally on two consecutive days with 10 ml of a virulent Mhyo strain. On SD 59 or 60 all animals were necropsied.

All piglets were observed daily after vaccination for clinical signs. Body temperatures were monitored as well as local reactions. The latter by palpation every second day, starting on the day of vaccination until 26 days post vaccination, or until local reactions had resolved. Serum samples were collected from all animals on the day of vaccination as well as on SD 22, 29 and 35. Samples were tested for antibodies against PCV2, Mhyo and Lawsonia (groups 3 and 5 only) and were compared with each other. At necropsy, the Mhyo specific lung lesions were scored.

Results

At the start of the experiment all animals were found to be healthy.

At the time of vaccination (SDO) all groups had comparable average rectal temperatures. At four hours post vaccination only a slight increase was observed in the average rectal temperatures for all treatment groups (average increase of 0.4-0.6° C.). The maximum temperature increase of 1.8° C. for one animal was observed in group 2. These temperature rises are acceptable for a swine vaccine.

In the vaccinated groups most of the animals had local reactions (80-100% of piglets), but the average size was low, viz. 2 cm for all vaccinated groups, including the positive control. The lowest percentage of animals having local reactions and the lowest average size could be found in Group 2, viz. 80% and 1.8 cm respectively. These figures are acceptable for vaccines authorised for use in practice.

Regarding PCV serologie, at the time of vaccination (SDO) all animals were negative for PCV2 IgM antibodies. Following vaccination no clear difference in average PCV2 total Ig antibody response could be observed between the test groups 1, 2 and 3. Three weeks post vaccination the percentage of PCV2 IgM positive animals was 93% in Groups 1 to 3 and 0% for the controls. This shows that the combination vaccines were able to actively induce an anti-ORF2 antibody titer.

Regarding Mhyo, at the start of the experiment none of the piglets were positive for antibodies against Mhyo. Four weeks post vaccination (SD30) in the test groups 1, 2 and 3 between 93 and 100% of the animals were Mhyo positive. In the positive control group this was 40%. Lung lesion scores confirmed a good efficacy against an Mhyo infection as depicted below in Table 2.

Regarding Lawsonia serology, at the start of the study all animals in groups 3 and 5 were negative for antibodies against Lawsonia. During the course of the study the percentage of positive animals in the LFD vaccinated group increased from 60% at SD30 to 100% at the end of the study. None of the animals in the control group had a Lawsonia serological response until the end of the study.

TABLE 2
Median Lung Lesions score and % of animals
with a low score (<5% lesions)

| Group | Median | <5% |
|---|---|---|
| 1. PCV/Mhyo, 0.5 PCVU Mhyo, Montanide | 1.2 | 80% |
| 2. PCV/Mhyo, 1 PCVU Mhyo, Montanide | 1.6 | 80% |
| 3. PCV/Mhyo, 1 PCVU Mhyo plus Lawsonia, Montanide | 1.6 | 72% |
| 4. Porcilis ® M Hyo ID ONCE, positive control | 0 | 85% |
| 5. No vaccination, negative control | 10.3 | 20% |

Study 4

Objective

The objective of the fourth study was to evaluate efficacy and safety of PCV2/Mhyo combination vaccines using a nano emulsion adjuvant as described in Study 3, to which combination vaccines live attenuated PRRS virus was added to arrive at a triple combination vaccine. The efficacy towards protection against infection with PCV2 is evaluated by assessing anti-ORF2 serology. The efficacy against infection with Mycoplasma hyopneumoniae is evaluated by comparing the serological response with that of the commercially available Mhyo vaccine Porcilis® Mhyo (MSD Animal Health, Boxmeer, The Netherlands). The efficacy against an infection with PRRS virus is evaluated by assessing the PRRs viraemia upon challenge with a pathogenic PRRS strain, 4 weeks post vaccination.

Experimental Design

The progeny of 10 sows was available for this study. A total of 40 animals were allotted to 4 groups of 10 piglets each. All animals were transferred to an animal facility when they were approximately 4 weeks old. Groups 1 to 4 were intradermally vaccinated using the IDAL® vaccinator into the right side of the neck. Groups 1 and 2 each received an ORF2 protein based PCV2 vaccine comprising in addition Mhyo bacterin (the same antigen as in the commercially available product Porcilis® M Hyo), in which combination vaccine a live PRRS virus vaccine (Porcilis PRRS) was reconstituted. The vaccine for group 1 was based on Montanide IMS 251, available from SEPPIC, France) to which 3% ovalbumin was added. The vaccine of group 2 contained the same adjuvant but no ovalbumin was added. Each vaccine contained 9 μg/dose of the ORF2 protein, and Mhyo antigen at 1-2 times the concentration of the M Hyo antigen in the commercially available vaccine Porcilis® M Hyo ID ONCE. The PRRS vaccine was a freeze-dried vaccine and was reconstituted immediately before administration to contain 10^{4.5} TCID₅₀ of virus per dose of 200 μl using the appropriate PCV2 vaccine or a diluent. Group 3 only received the PRRS vaccine and group 4 remained unvaccinated and served as control. All piglets were observed daily for clinical signs. The animals were challenge-infected with pathogenic PRRS virus (type I) when they were approximately 8 weeks old (day 28). The challenge material contained (a calculated dose of) 5.3 log 10 TCID50 of the virus in 2 ml. The material was intra-nasally administered, 1 ml per nostril. At the end of the observation period (49 days after vaccination corresponding to 21 days post challenge) all pigs were sacrificed. Blood samples (via v. jugularis) will be were taken from all animals individually on day 0, 14, 28 (right before challenge), 31, 35, 38, 42 and 49 and tested for the presence of PRRS virus, for antibodies against PRRSV, PCV2 and Mhyo.

Results

No animals showed any clinical signs due to vaccination and rectal temperatures remained within 1.5° C. from controls. The vaccines are thus regarded safe.

Regarding Mhyo, the serological response of the combination vaccine appears to be comparable to that as obtainable with the commercially available vaccine Porcilis M Hyo (no numerical results depicted in a figure). It may thus be assumed that the vaccine protects against infection with Mhyo.

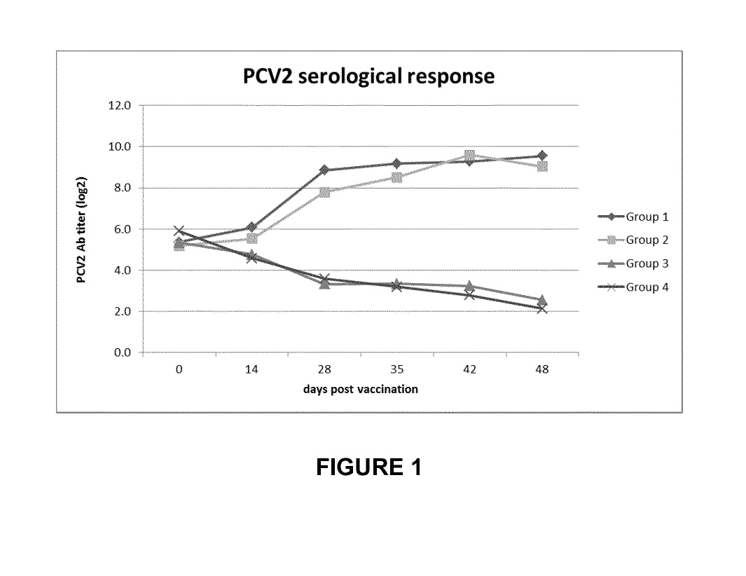

The results of the PCV2 serological response are given in FIG. 1. It appears that the two combination vaccines induce a positive anti-ORF2 antibody response which means that the vaccines induce protection against infection with wild-type PCV2.

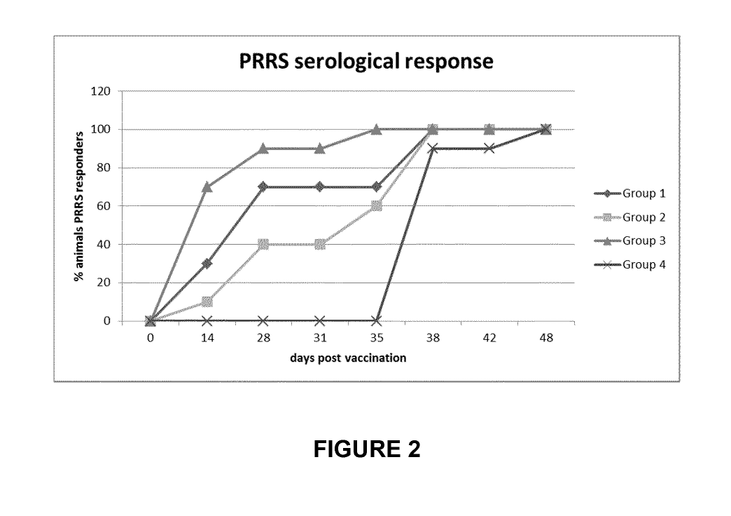

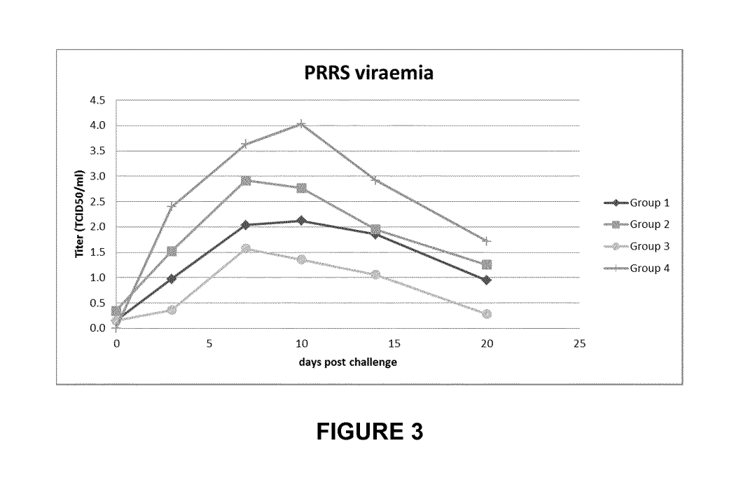

The results of the PRRS serological response are given in FIG. 2. It appears that the two combination vaccines, like the commercially available PRRS vaccine, induce a positive anti-PRRS antibody response before challenge. This is an indication that the vaccines provide protection against PRRS virus infection. In FIG. 3 the viraemia data are given. It appears that all three vaccines provide protection against PRRS virus infection since viraemia levels are lower than the level in the positive control animals (group 4) at each point in time.

Study 5

The objective of the fifth study was to evaluate efficacy and safety of PCV2/Mhyo combination vaccines using alternative nano emulsion adjuvants, when compared to the nano emulsion adjuvant as described in Study 3. Regarding PCV2, the efficacy was evaluated by assessing anti-ORF2 serology. Regarding Mycoplasma hyopneumoniae, efficacy was evaluated by comparing the serological response with that of the commercially available Mhyo vaccine Porcilis® M Hyo ID ONCE (MSD Animal Health, Boxmeer, The Netherlands).

The experimental design was largely the same as described here above. In particular, a total of 50 piglets were allotted to 5 treatment groups: 5 groups of 10 piglets each. The piglets were vaccinated intradermally when they were approximately three weeks old. Piglets from groups 1 to 3 were vaccinated with a single dose of vaccine (0.2 ml) formulated with PCV2-Mhyo antigen as used in Study 3 and three different adjuvants based on nano emulsions of mineral oils in water (the fact that the emulsions were actually nano emulsions was established microscopically). The first adjuvant (received by group 1) was formulated as an Amphigen® like nano-emulsion, viz. the same adjuvant emulsion as Amphigen® (obtainable from Zoetis), but containing the mineral oil as a nano emulsion by subjecting the adjuvant to microfluidisation. The second adjuvant (received by group 2) was formulated as a Metastim® (Boehringer Ingelheim) like adjuvant, made by adding vitamin E-acetate to the commercially available adjuvant and emulsifying the adjuvant to become a nano-emulsion by microfluidisation. The third adjuvant (received by Group 3) resembled IMS251 as used in Example 3, apart from the fact that vitamin E-acetate was added, and the method of obtaining the emulsion was different, namely via an aqueous intermediate emulsion (i.e. an aqueous concentrate to be used for formulating the vaccine), instead of a pure oil/surfactant intermediate (i.e. an oily concentrate to be used to formulate the vaccine). Piglets from group 4 were vaccinated with Porcilis M Hyo ID ONCE. Piglets from group 5 were not vaccinated.

The results indicated that the three experimental vaccines based on alternative nano-emulsions of mineral oil in water were completely safe: no increase in body temperature was noticed after vaccination and the average local reactions were less than or equal to the average local reactions as observed with the commercial vaccine Porcilis M Hyo ID ONCE. PCV2 serology showed that in all three groups receiving the combined PCV2/Mhyo vaccine (groups 1-3) the anti-ORF2 titer increased due to vaccination, whereas in groups 4 and 5 a continuous decrease was seen in the same period. Regarding Mhyo, all animals (groups 1-5) were challenged. The percentage of positive animals after challenge was 90-100% in groups 1-3, the same as in the positive control group 4 (receiving the commercial M Hyo vaccine). In the negative control group 5 the percentage of anti-body positive animals was 0%.

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. A vaccine comprising in combination a non-replicating immunogen of porcine circovirus type 2 (PCV2), a non-replicating immunogen of Mycoplasma hyopneumoniae, and an adjuvant comprising a nano-emulsion of mineral oil in water, for use in prophylactically treating an animal against an infection of PCV2, Mycoplasma hyopneumoniae, or both PCV2 and Mycoplasma hyopneumoniae, wherein the vaccine is administered into the dermis of the animal.

17. A vaccine of claim 16, wherein the vaccine is administered by a single dose.

18. A vaccine of claim 16, wherein the vaccine is administered with a needle-less vaccination device.

19. A vaccine of claim 16, wherein the non-replicating immunogen of PCV2 is recombinantly expressed ORF2 protein of PCV2.

20. A vaccine of claim 16, wherein the non-replicating immunogen of PCV2 is baculovirus expressed ORF2 protein of PCV2.

21. A vaccine of claim 16, wherein the non-replicating immunogen of Mycoplasma hyopneumoniae is a bacterin.

22. A vaccine of claim 16, wherein the vaccine additionally comprises live attenuated porcine reproductive and respiratory syndrome (PRRS) virus.

23. A vaccine of claim 22, wherein the live attenuated PRRS virus is combined with the immunogen of PCV2 and Mycoplasma hyopneumoniae within 24 hours before administration.

24. A vaccine of claim 23, wherein the live attenuated PRRS virus is combined with the immunogen of PCV2 and Mycoplasma hyopneumoniae within 6 hours before administration.

25. A vaccine of claim 16, wherein the vaccine additionally comprises non-replicating immunogen of Lawsonia intracellularis.

26. A vaccine of claim 25, wherein the immunogen of Lawsonia intracellularis is combined with the immunogen of PCV2 and Mycoplasma hyopneumoniae within 24 hours before administration.

27. A vaccine of claim 26, wherein the immunogen of Lawsonia intracellularis is combined with the immunogen of PCV2 and Mycoplasma hyopneumoniae within 6 hours before administration.

28. A vaccine of claim 25, wherein the immunogen of Lawsonia intracellularis is added to the vaccine in the form of a freeze-dried composition of Lawsonia intracellularis bacterin.

29. A method for prophylactically treating an animal against an infection with porcine circovirus type 2 (PCV2), an infection with Mycoplasma hyopneumoniae, or an infection of both PCV2 and Mycoplasma hyopneumoniae, by intradermally administrating to the animal a vaccine comprising in combination non-replicating immunogen of PCV2, non-replicating immunogen of Mycoplasma hyopneumoniae, and an adjuvant containing a nano-emulsion of mineral oil in water.

30. A method of manufacturing a vaccine comprising the non-replicating immunogen of porcine circovirus type 2 (PCV2), the non-replicating immunogen of Mycoplasma hyopneumoniae, and an adjuvant containing a nano-emulsion of mineral oil in water, for intradermal administration to an animal to prophylactically treat the animal against an infection with PCV2, an infection with Mycoplasma hyopneumoniae, or an infection of both PCV2 and Mycoplasma hyopneumoniae.
